(19) United States
(12) Patent Application Publication
(10) Pub. No.: US 2025/0295334 A1
(43) Pub. Date: Sep. 25, 2025

(54) BLOOD MONITORING SYSTEMS AND METHODS

(71) Applicant: Terumo Cardiovascular Systems Corporation, Ann Arbor, MI (US)

(72) Inventors: Joseph William McElroy, Ann Arbor, MI (US); David Frederick Lemmerhirt, Saline, MI (US); Wessam Rafic Bazzi, Northville, MI (US); Catherine Hook-Morgan Leonard, Tecumseh, MI (US); Honglei Liu, Ann Arbor, MI (US)

(21) Appl. No.: 19/081,450

(22) Filed: Mar. 17, 2025

Related U.S. Application Data
(60) Provisional application No. 63/567,278, filed on Mar. 19, 2024.

(51) Int. Cl. A61B 5/145; A61B 5/00; A61M 1/36

(52) U.S. Cl. CPC A61B 5/14542; A61B 5/6866; A61B 2505/05; A61B 2560/0223; A61M 1/3666

(57) ABSTRACT

Medical systems can be used to monitor various types of patient parameters. For example, this document describes patient parameter monitoring systems that obtain and display information to provide continuous, in-line monitoring of various patient parameters during medical procedures, such as parameters of blood contained within an extracorporeal perfusion circuit during an open heart surgery procedure.

### CROSS-REFERENCE TO RELATED APPLICATIONS

This application claims the benefit of U.S. Provisional Application Ser. No. 63/567,278 filed Mar. 19, 2024. The disclosure of the prior application is considered part of (and is incorporated by reference in) the disclosure of this application.

### BACKGROUND

### 1. Technical Field

This document relates to medical systems for monitoring patient parameters and methods for their use. For example, this document relates to patient parameter monitoring systems that obtain and display information to provide continuous, in-line monitoring of various patient parameters during medical procedures, such as parameters of a patient's blood contained within an extracorporeal perfusion circuit.

### 2. Background Information

Continuous in-line monitoring during cardiopulmonary bypass surgery is a critical component of perfusion safety and improving patient outcomes. Studies have shown that appropriate regulation of blood gas parameters is essential to avoid the negative outcomes linked to sub-optimal blood gas parameter control.

### SUMMARY

This document describes medical systems for monitoring patient parameters and methods for their use. For example, this document describes patient parameter monitoring systems that obtain and display information to provide continuous, in-line monitoring of various patient parameters during medical procedures, such as parameters of a patient's blood contained within an extracorporeal perfusion circuit.

In one aspect, a patient parameter monitoring system includes a calibration system that is described herein. The calibration system uses a two-point gas calibration process to improve the accuracy of blood parameter measurement during cardiopulmonary bypass. The calibration process conserves gas by employing one or more of the following strategies: (i) the system continuously or periodically monitors output signals from blood parameter sensors during calibration and ceases a flow of calibration gas when one or more of the sensor signal intensities approaches equilibrium; (ii) self-diagnostic checks are integrated into the calibration process in a way that minimizes consumption of gas beyond that which is needed to properly calibrate each sensor, the gas calibrator assures consistent operation by conducting self-diagnostics including one or more of leak rate checks, valve operation checks, and measuring flow rate to the sensor(s) being calibrated; and (iii) a gas calibration device employs an adjustable pressure regulator and a fixed orifice to establish a repeatable and accurate flow rate of calibration gas to one or more sensors being calibrated.

In another aspect, a blood parameter monitoring system provides to the clinician a single interface that integrates a variety of information to represent the hemodynamic and metabolic state of the patient through direct measurement, acquisition, and calculation of relevant parameters and displaying that information via numeric and graphical readings. By providing this information in a configurable manner, the clinician can optimize the display of information to highlight the most critical information and associations, through the use of a configurable numeric display and customizable graphs. The numeric display allows for optimizing the presentation of the parameter information through both the position on the screen, the proximity of given parameters to each other, and the size of the information display. Similarly, the graphing allows for parameters to be associated on the same graphing screen to enable the visual identification of associations between parameter trends over time.

In another aspect, a blood parameter monitoring system calculates the partial pressure of oxygen (“PO₂”) based on reverse calculating a mathematical model. The calculator is unique in utilizing in-line continuous measurements from arterial and venous sides of the device during cardiopulmonary bypass to execute the calculations. The calculator enables the user to have PO₂ measurements available even without having a blood parameter monitoring (“BPM”) connected on the venous side. The Calculator uses the continuous in-line arterial pH and temperature measurements from BPM probe, and venous SO₂ from a hematocrit/saturation (“HSAT”) probe. Calculated PO₂ generates accurate PO₂ level relative to the measured PO₂ levels. Calculated PO₂ will allow to have more accurate calculations of other calculated parameters as oxygen consumption and oxygen extraction equations. The equation is derived from Siggaard Anderson's mathematical model. It is reversed using Newton's Raphson iteration mathematical model.

In another aspect, a blood parameter monitoring probe is described herein. The blood parameter monitoring probe includes a housing and a spring-backed pivoting clip device that is releasably coupled to the housing.

In another aspect, a calibration device for a blood parameter monitoring system is described herein. The calibration device includes a color chip carrier assembly has a built-in magnet that, when a HSAT probe is connected, is used to trigger hall effect sensor magnets within the HSAT probe.

In another aspect, a color-coded helix-shaped flexible marking apparatus (“marker”) used to visually demarcate particular cables and associated devices is described herein. The marker is configured to be manually attached and detached (without tools) from a cable, wire, tube, hose, etc. An internal contour of the helical shape is a circular cut-out with a diameter that is narrowest at a longitudinal center of the marker, the diameter expands outward to a maximum at each end of the marker.

In another aspect, a BPM probe is described herein. The BPM probe includes a housing that has an ergonomic two finger loop for handling the BPM.

In another aspect, a patient parameter monitoring system is described herein. The patient parameter monitoring system includes a HSAT probe that includes three LEDs to illuminate flowing blood and a photodetector to measure the reflectance of light off the blood. The HSAT probe includes a sapphire window to interface with a disposable to ensure high durability and high efficiency optical transmission. The HSAT probe also includes a light barrier that separates the LEDs and the photodetector.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic illustration of an example medical procedure that uses a blood parameter monitoring system in accordance with some embodiments.

FIG. 2 is an illustration of an example blood parameter monitoring system in accordance with some embodiments.

FIG. 3 is a perspective view of a portion of a probe device that can be used with the blood parameter monitoring systems described herein.

FIG. 4 is a removable clip portion of the probe device of FIG. 3.

FIG. 5 is a cross-sectional view of the removable clip portion of FIG. 4.

FIG. 6 is a perspective view showing a cable marker in accordance with some embodiments.

FIG. 7 is a cross-sectional view of the arrangement of FIG. 6.

FIG. 8 is a perspective view of a calibrator system of the blood parameter monitoring systems described herein.

FIG. 9 is a time-based graph showing features of the calibration process that can be performed by the calibrator system of FIG. 8.

FIG. 10 is a process flowchart showing features of the calibration process that can be performed by the calibrator system of FIG. 8.

Like reference numbers represent corresponding parts throughout.

### DETAILED DESCRIPTION

This document describes medical systems for monitoring patient parameters and methods for their use. For example, this document describes patient parameter monitoring systems that obtain and display information to provide continuous, in-line monitoring of various patient parameters during medical procedures, such as parameters of a patient's blood contained within an extracorporeal perfusion circuit.

While the systems for monitoring patient parameters described herein are primarily described in the context of an open-heart surgery procedure using a heart/lung bypass machine, it should be understood that the open-heart surgery procedure using a heart/lung bypass machine is just an example. The innovative concepts regarding the patient parameter monitoring systems described in the context of the open-heart surgery procedure using medical systems extend, without limitation, to various other types of medical procedures using other medical treatment systems in conjunction with other devices/systems.

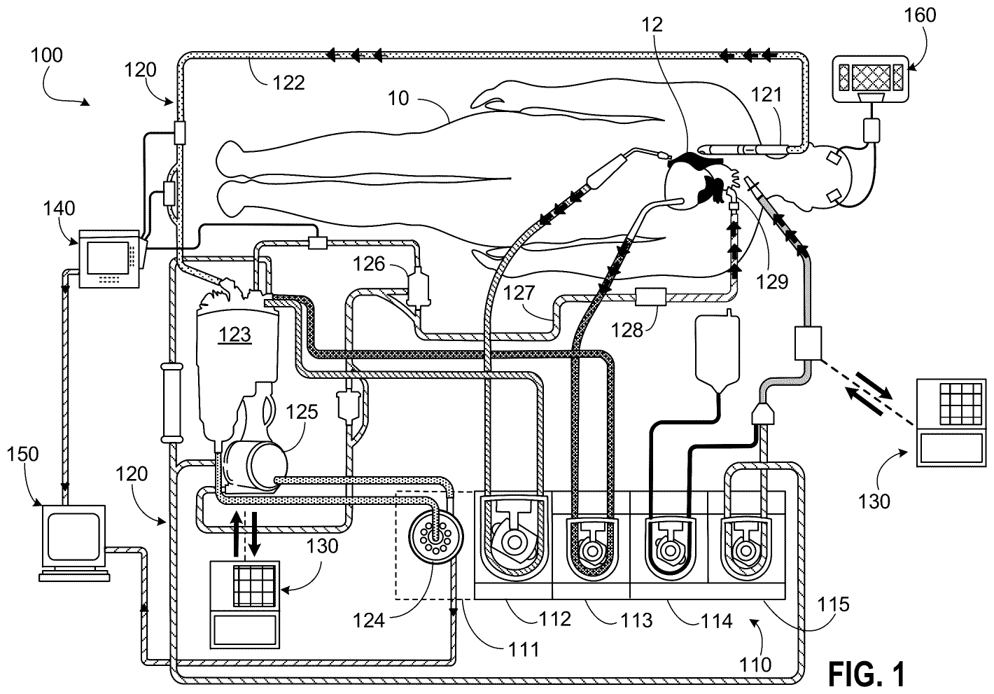

As shown in the example of FIG. 1, various types of medical procedures can be performed on a patient 10 while the patient 10 is connected to a life-sustaining heart/lung bypass machine system 100. In this example, the patient 10 is undergoing open-heart surgery during which the heart 12 and lungs of the patient 10 are temporarily intentionally caused to cease functioning. Because the body of the patient 10 continues to have a metabolic need to receive a supply of circulating oxygenated blood during the medical procedure, however, the heart/lung bypass machine system 100 performs such functions. That is, as described further below, the heart/lung bypass machine system 100 is connected to the patient 10 and performs the functions of the heart 12 and lungs of the patient 10 so that the patient 10 stays alive and healthy during open-heart surgery.

The heart/lung bypass machine system 100 can be used for many different types of medical procedures. For example, the medical procedures for which the heart/lung bypass machine system 100 can be used include, but are not limited to, coronary artery bypass grafts, heart valve repairs, heart valve replacements, heart transplants, lung transplants, ablation procedures, repair of septal defects, repair of congenital heart defects, repair of aneurysms, pulmonary endarterectomy, pulmonary thrombectomy, and the like.

In the depicted example, the heart/lung bypass machine system 100 includes components and sub-systems such as a heart/lung machine 110, an extracorporeal circuit 120, one or more temperature control systems 130, a blood monitoring system 140, a perfusion data management system 150, and a regional oximetry system 160. Some types of procedures that use the heart/lung bypass machine system 100 may not require all of the components and sub-systems that are shown. Some types of procedures that use the heart/lung bypass machine system 100 may require additional components, monitors, and/or sub-systems that are not shown.

The extracorporeal circuit 120 is connected to the patient 10, and to the heart/lung machine 110. Other systems, such as the temperature control system 130, blood monitoring system 140 (e.g., a CDI® blood gas monitor made by Terumo Cardiovascular Corporation), and perfusion data management system 150 may also be arranged to interface with the extracorporeal circuit 120. The extracorporeal circuit 120 is connected to the patient 10 at the patient's heart 12. Oxygen-depleted blood (venous blood) from the patient 10 is extracted from the patient 10 at the patient's heart 12 using a venous catheter 121. As described further below, the blood is circulated through the extracorporeal circuit 120 to receive oxygen and remove carbon dioxide. The oxygenated blood is then returned through the extracorporeal circuit 120 to the patient's heart 12 via an aortic cannula 129.

The extracorporeal circuit 120 can include, at least, a venous tube 122 (e.g., lining, tubing) that is coupled to the venous catheter 121, a blood reservoir 123, a centrifugal pump 124, an oxygenator 125, an arterial filter 126, one or more air bubble detectors 128, and an arterial tube 127 (e.g., lining, tubing) that is coupled to the aortic cannula 129. The venous catheter 121 and venous tube 122 are in fluid communication with the venous side of the circulatory system of the patient 10. The venous tube 122 is also in fluid communication with an inlet to the reservoir 123. An outlet from the reservoir 123 is connected by tubing to an inlet of the pump 124. The outlet of the pump 124 is connected by tubing to an inlet of the oxygenator 125. The outlet of the oxygenator 125 is connected by tubing to an inlet of the arterial filter 126. An outlet of the arterial filter 126 is connected to the arterial tube 127. One or more pressure transducers (not depicted) can be located along the arterial tube 127 to detect a heart/lung machine (HLM) system line pressure of the blood in the arterial tube 127, which is measured by the heart/lung machine 110 and monitored by the perfusionist. The arterial tube 127 is connected to the arterial cannula 129, which is in physical contact with the heart 12 and in fluid communication with the arterial side of the circulatory system of the patient 10.

Briefly, the extracorporeal circuit 120 operates by removing venous, oxygen-depleted blood from the patient 10 via the venous catheter 121 and depositing the venous blood in the reservoir 123 via the venous tube 122. Moreover, one or more air bubble detectors 128 can be located at various sites along the extracorporeal circuit 120. Blood from the reservoir 123 is drawn from the reservoir 123 by the pump 124. While the depicted embodiment includes a one-time use centrifugal pump as the pump 124, in some cases a peristaltic pump of the heart/lung machine 110 is used instead. The pressure generated by the pump 124 propels the blood through the oxygenator 125. In the oxygenator 125, the venous blood is enriched with oxygen, and carbon dioxide is removed from the blood. The now oxygen-rich arterial blood exits the oxygenator 125, travels through the arterial filter 126 to remove emboli, and is injected into the patient's heart 12 through the arterial tube 127 via the aortic cannula 129.

The heart/lung bypass machine system 100 also includes the heart/lung machine 110. The heart/lung machine 110 is a complex system that includes multiple pumps, monitors, controls, user interfaces, alarms, safety devices, and the like, that are all monitored and operated/adjusted by the perfusionist during a surgical procedure. For example, the depicted heart/lung machine 110 includes an arterial pump 111 (which can be a drive system for a disposable centrifugal pump 124 as shown, or a peristaltic pump), a suction pump 112, a vent/drainage pump 113, a cardioplegia solution pump 114, and a cardioplegia delivery pump 115. The heart/lung machine 110 can also include, or be interfaced with, devices such as a tubing occluder, gas blender, and the like.

The heart/lung bypass machine system 100 also includes one or more temperature control systems 130. In a first aspect, the temperature control system(s) 130 is/are used to heat and cool the patient's blood in the oxygenator 125 via a heat exchanger. Additionally, the temperature control system(s) 130 is/are used to heat and cool the cardioplegia solution being delivered to the heart 12 of the patient 10. In general, the temperature control system(s) 130 is/are used in cooling modes during the procedure (to reduce metabolic demands), and subsequently used to warm the blood and/or cardioplegia solution when the surgical procedure is nearing its end.

The heart/lung bypass machine system 100, as depicted, also includes the perfusion data management system 150 and the regional oximetry system 160. These systems can also be used by the perfusionist to monitor the status of the patient 10 and/or the status of the heart/lung bypass machine system 100 during surgical procedures.

The heart/lung bypass machine system 100, as depicted, also includes the blood monitoring system 140. The blood monitoring system 140 is used to monitor the venous and/or arterial extracorporeal blood of the patient 10 during the surgical procedure. In some cases, the perfusionist will need to adjust other components or subsystems of the heart/lung bypass machine system 100 in response to readings from the blood monitoring system 140.

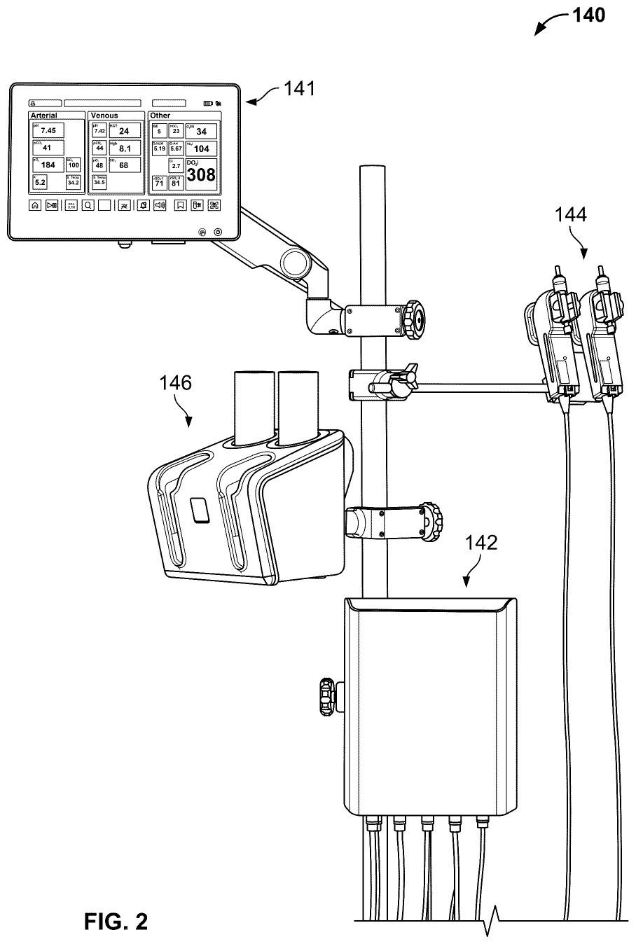

FIG. 2 provides a more detail illustration of the example blood monitoring system 140. In the depicted embodiment, the blood monitoring system 140 includes a touchscreen display 141, a processing core 142, one or more probes 144 (e.g., arterial BPM probe, venous BPM probe, and/or HSAT probe), and a calibrator 146. These subsystems function together to provide blood monitoring of a patient.

The patient parameters that can be monitored by the blood monitoring system 140 can include, but are not limited to, potential of hydrogen (pH), partial pressure of carbon dioxide (pCO₂), partial pressure of oxygen (pO₂), potassium ion (K+), oxygen saturation (SO2), hematocrit (HCT), hemoglobin (Hgb), blood flow rate, cardiac index (CI), base excess (BE), bicarbonate, oxygen consumption, indexed oxygen consumption (VO₂i), oxygen delivery (DO₂), indexed oxygen delivery (DO₂i), cerebral regional oxygen saturation (rSO₂), oxygen extraction ratio (O₂ER), body surface area (BSA), and shunt sensor temperature.

The blood monitoring system 140 (also referred to as the “CDI OneView System”) is an AC-powered, microprocessor-based system. The blood monitoring system 140 uses an optical fluorescence technology to measure blood gases, pH and potassium. It additionally uses an optical reflectance technology to measure oxygen saturation, hematocrit and hemoglobin. Optical fluorescence measurements are taken through a blood parameter module (BPM) probe 144 connected to a disposable shunt sensor. Optical reflectance measurements are taken through the hematocrit/saturation (“HSAT”) probe 144 connected to a disposable cuvette. Shunt sensors and cuvettes are incorporated in the extracorporeal circuit (FIG. 1). Flow measurements are ascertained using one or more flow sensors (not shown) attached to tubing of the extracorporeal circuit.

The processing core 142 provides power to all connected modules and module-to-module communication, such as between the calibrator 146 and the one or more probes 144. Any calculated parameter that requires input from more than one module is calculated in the processing core 142 and sent to the touchscreen display 141. The processing core 142 has a 25-minute battery backup for use during transport or for emergency power.

As described further below, the blood monitoring system 140 provides to the clinician a single user interface by the touchscreen display 141. The touchscreen display 141 can be user-configured to integrates a variety of information to represent the hemodynamic and metabolic state of the patient through direct measurement, acquisition, and calculation of relevant parameters and displaying that information via numeric and graphical readings on the touchscreen display 141. By providing this information in a flexibly configurable manner on the touchscreen display 141, the clinician can optimize the display of information, as desired, to highlight the most critical information and associations through the use of a configurable numeric display and customizable graphs. The touchscreen display 141 allows for optimizing the presentation of the parameter information through user-configuration of both the positions on the screen, the proximity of given parameters to each other, and the sizes of the information/parameters being displayed. Similarly, the parametric graphing on the touchscreen display 141 allows for parameters to be associated on the same graphing screen to enable the clinician use to ascertain visual associations between parameter reading and trends over time.

The one or more BPM and HSAT probes 144 can be flexibly used for performing blood parameter measurements on either the arterial or venous side of an extracorporeal circuit (FIG. 1). Which side of the extracorporeal circuit is being measured can affect the ranges and alarm levels of the measurements. In addition, the user needs to be able to ensure the probe 144 is placed on the appropriate side of the circuit. In prior blood monitoring systems, these aspects were accomplished by having dedicated probes for arterial or venous. The blood monitoring system 140 described herein incorporates a universal version of each probe 144 type (BPM and HSAT), which can be connected to any port on the processing core 142 and used on either side. By assigning a role to each probe of the one or more probes 144, it will be configured by the processing core 142 to have the appropriate ranges as well as provide an indicator via the touchscreen display 141 to signal to the clinician user the assigned role (arterial or venous). For use applications of the blood monitoring system 140 beyond cardiopulmonary bypass, additional roles can be identified and so configured.

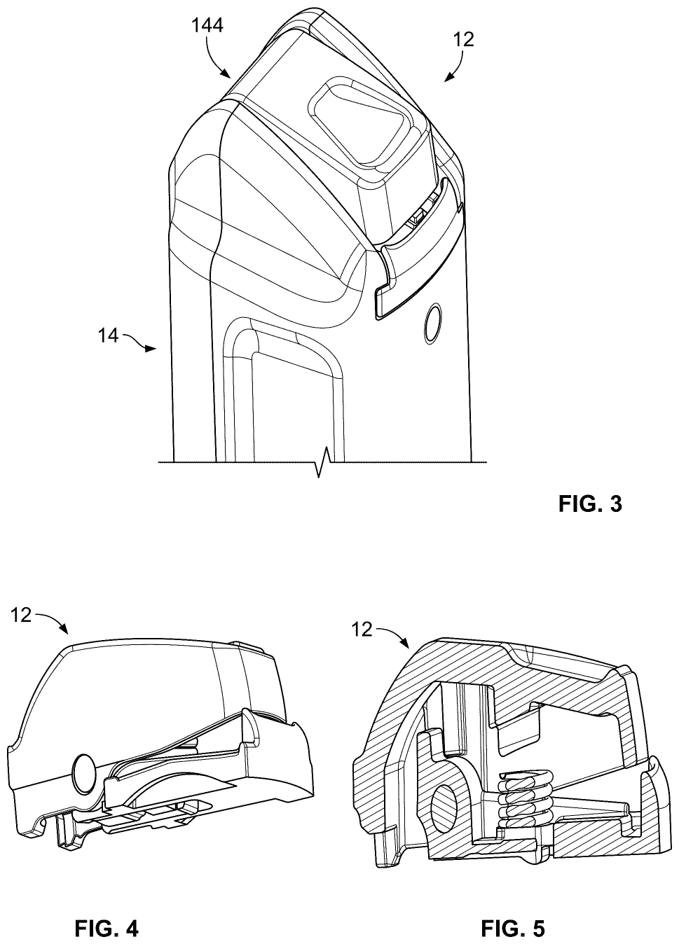

Referring also to FIGS. 3-5, the HSAT probe 144 uses a spring-backed, pivoting clip 12 to lock onto and release from a mating barbed clip on a color chip carrier assembly that is used for calibration of the HSAT probe 144. The illustrated spring-backed, pivoting clip 12 (shown in isolation in FIGS. 4 and 5) is releasably coupled to the housing 14 of the HSAT probe 144 using a tongue and groove arrangement so that a user can slide the clip 12 into and out of the housing 14 of the HSAT probe 144 to facilitate cleaning and/or replacement, if needed. The spring clip assembly 12 is designed to hold all the parts together so that no individual part gets lost. It is also designed to require a higher force to remove from the housing 14 of the HSAT probe 144 than it experiences during engagement with the blood loop cuvette or the color chip carrier assembly.

In some embodiments, the color chip carrier assembly (which is used to calibrate an HSAT probe 144) has a built-in magnet that, when an HSAT probe 144 is connected, triggers hall effect sensor magnets within the HSAT probe 144. The probe diagnostics initiated at startup of the HSAT probe 144 determine the health of the three internal magnets by determining if all three hall effect sensors are triggered by the internal magnet of the color chip carrier. The design of the color chip carrier ensures a large enough magnet to encompass all three hall effect sensors with a strength capable of triggering the sensors.

As shown and described elsewhere herein, the color chip that is used for calibration of the HSAT probe 144 is designed to be an extremely stable reference material that fits within the color chip carrier assembly to ensure a specific reflectance in order to assess HSAT probe optical health. The design of the color chip enables any HSAT probe 144 to be used in pair with any color chip to assess optical health.

In some embodiments, the optical module assembly of the HSAT probe 144 includes three LEDs to illuminate flowing blood and a photodetector to measure the reflectance of the light off the blood in enable the calculation of HCT, Hgb, and SO2. The HSAT probe 144 uses a Sapphire window to interface with the disposable to ensure high durability and high efficiency optical transmission. The LEDs and photodetectors of the HSAT probe 144 are separated by a light barrier that ensures the light measured by the photodetector is the light reflected from the blood. The HSAT probe 144 comprises two optical windows (one for LEDs, one for photodetector). In some embodiments, additional optical windows can be added to measure effects of flow to the measurement.

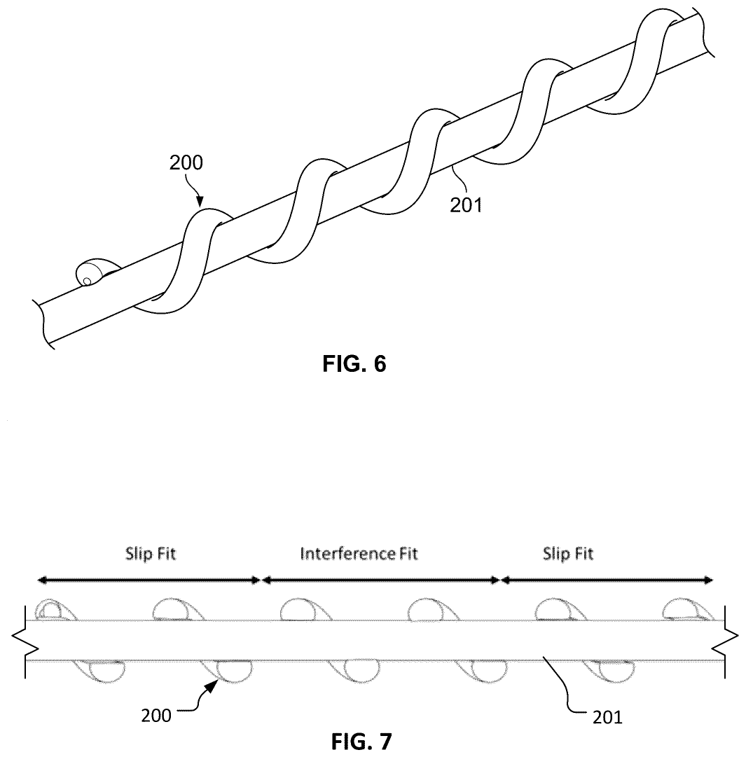

Referring also to FIGS. 6 and 7, a color-coded helix-shaped flexible apparatus 200 (“marker 200”) shaped similar to cavatappi pasta can be used to visually demarcate particular cables 201 and associated devices. The marker 200 can be manually attached and detached (without tools) from a cable 201 (or wire, tube, hose, etc.) for the purpose of providing a visual cue for identification. The design of the marker 200 is such that there are no small pathogen-harboring crevices formed between the turns of the helical structure, making it ideal for use in sterile settings. The internal contour of the helical shape of the marker 200 is a circular cut-out, with a diameter that is narrowest at the longitudinal center of the marker 200. The diameter expands (i.e., drafts) outward to a maximum at each end of the marker 200. The narrowest portion in the middle region of the marker 200 is sized to form an interference fit with the cable 201 (or other part) to which it is attached. The widest outer portions of the marker 200 are meant to form a loose slip fit with the cable 201 (or other part) to which it is attached. This is intended to make it easy to attach and detach the marker 200 to the cable 201 without tools, while providing and maintaining a sufficiently firm grip by the marker 200 on the cable 201 (or other part).

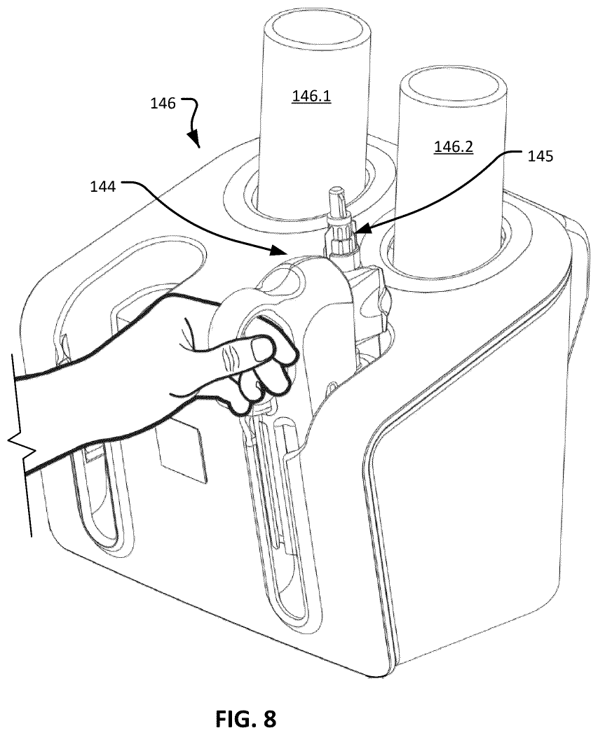

As previously described in reference to FIG. 2, the blood monitoring system 140 includes the calibrator 146. Referring to FIG. 8, the calibrator 146 is shown in greater detail. The purpose of the calibrator 146 is to calibrate up to two BPM probes 144 in conjunction with particular disposable shunt sensors 145. As described further below, the shunt sensors 145 are used for measuring pH, pCO₂, and pO₂ and are calibrated using a two-point tonometered calibration approach, similar to that used to calibrate the electrodes in laboratory analyzers.

The calibration process uses the calibrator 146 and two canisters of calibration gases, i.e., gas 146.1 and gas 146.2. Each gas canister (gas 146.1 and gas 146.2) contains a particular mixture/composition of calibration gases (e.g., carbon dioxide (CO₂) and oxygen (O₂)). For example, the canister of gas 146.1 can contain: 7.5+/−0.1% of CO₂; 24+/−0.2% of O₂; and the balance of N₂. In contrast, the canister of gas 146.2 can contain: 2.8+/−0.1% of CO₂; 4.0+/−0.2% of O₂; and the balance of N₂. The calibration gas flow rates in the calibrator 146 are set at controlled and consistent levels by applying a regulated constant pressure to the inlet of a fixed orifice (as described further below).

As illustrated in FIG. 8, during calibration the shunt sensors 145 (attached to the BPM probes 144) are placed in the calibrator 146 to allow the calibration gases of gas 146.1 and gas 146.2 to flow through a buffer solution contained in each shunt sensor 145. This exposes the microsensors in each shunt sensor 145 to the gases with the known CO₂ and O₂ values/contents.

To perform the calibration, the blood monitoring system 140 measures the fluorescent intensities emitted by the microsensor in each shunt sensor 145 as it is exposed to first to gas 146.1 and then afterwards to gas 146.2. The blood monitoring system 140 then plots the two fluorescent measurements as a function of the predefined/known values/contents of the calibration gases 146.1 and 146.2. The blood monitoring system 140 uses the two points to create a slope and a y-intercept for that parameter. During the use of the blood monitoring system 140 (e.g., during a cardiopulmonary bypass procedure), as the blood monitoring system 140 measures the fluorescent intensity of the blood in the extracorporeal circuit, it uses the slope and intercept to extrapolate corresponding blood parameter values.

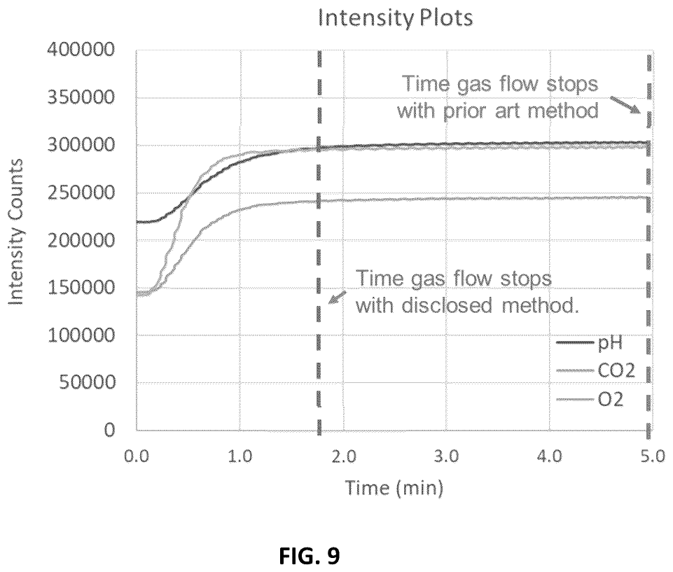

FIG. 9 illustrates the use of the two-point gas calibration routine to improve the accuracy of blood parameter measurement. Advantageously, the calibration process conserves gas by employing one or more of the following strategies.

First, the blood monitoring system 140 continuously or periodically monitors the output signals from the probes 144 during the calibration process and ceases the flow of calibration gas when one or more of the sensor signal intensities approaches or reaches signal intensity equilibrium. The definition of reaching signal intensity equilibrium can be, in some examples, that the signal intensity changes by less than a threshold amount over a set period of time. The threshold amount and the period of time parameters can be programmed into the software of the processing core 142.

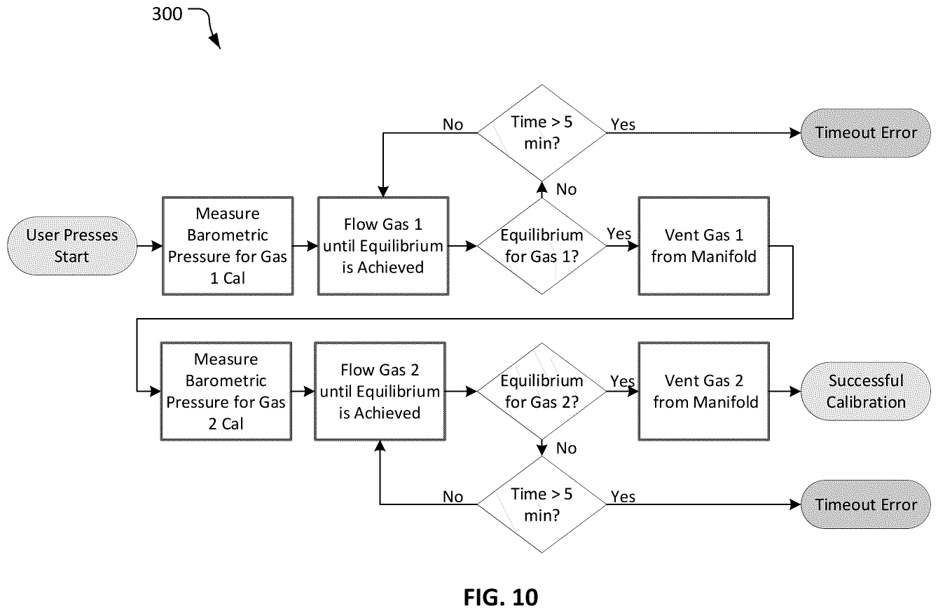

The process 300 shown in the flowchart of FIG. 10 also illustrates this feature of the calibration process. Actively monitoring for sensor signal equilibrium allows the blood monitoring system 140 to avoid wasting gas by continuing to flow gas once the output signal has reached a sensor signal equilibrium/settling point.

In contrast, conventional blood parameter measurement systems default initial gas flow duration with one additional fixed time allotment if equilibrium is not reached at a pre-determined checkpoint. This means that nearly every calibration run consumes more gas than is required for effective calibration. This means that fewer shunt sensors 145 can be calibrated from the canisters of calibration gases (i.e., gas 146.1 and gas 146.2). Or, the overall calibrator system must be larger to support the same number of calibrations.

Second, the blood monitoring system 140 (using the calibrator 146 as shown in FIG. 8) can perform self-diagnostic checks at the start of the calibration cycle in a way that minimizes the consumption of calibration gases (i.e., gas 146.1 and gas 146.2) beyond that which is needed to properly calibrate each shunt sensor 145. Such self-diagnostic checks of the calibrator 146 can be automatically initiated and run prior to the gas-consuming calibration process and in response to the start of the calibration process by a clinician user using the touchscreen display 141. For example, in some embodiments the gas calibrator 146 assures consistent operation by conducting self-diagnostics that include, but are not limited to, leak rate checks, valve operation checks, and measuring flow rate to the sensor(s) 145 being calibrated. Most of these tests do not consume gas; however, checking the flow rate requires that gas be flowing. The flow rate check is advantageous because it ensures that the calibrator 146 is operating properly and that the flow pathway to the sensor 145 is not occluded or diverted.

The calibration method disclosed herein estimates the flow rate while gas is flowing to the sensor 145 during calibration, for example during the first 60 seconds after the flow rate has stabilized. This means that the gas expended during the flow-rate measurement period contributes directly to reaching the equilibrium state needed for the calibration to be completed. In some embodiments, the gas flow rate is estimated by measuring the pressure of the calibration gas bottle (i.e., gas 146.1 and gas 146.2) when the flow rate has stabilized and again after a designated period (e.g., about 60 seconds). The flow rate is computed using the measured pressure drop and elapsed time, combined with the measured barometric pressure and the known volume of the gas bottle and manifold tubing. In some embodiments, the pressure measurement signals may be time-averaged to improve the resolution of the computed flow rate.

In contrast, conventional systems measure the flow rate at start-up or before or after the calibration process, which expends gas beyond that which is needed for calibration. Alternatively, one or more flow meters may be integrated directly in line with the gas flow path, which, however, adds cost and system size and complexity.

Third, in some embodiments described herein that use a two-point gas calibration routine to improve the accuracy of blood parameter measurement during cardiopulmonary bypass, the calibrator 146 employs an adjustable pressure regulator and a fixed orifice to establish a repeatable and accurate flow rate of calibration gas to one or more shunt sensors 145. The regulator set point and the orifice size are selected such that the velocity of the gas flowing through the orifice reaches the speed of sound. This condition, known as “choked flow” or “critical flow” occurs with a fixed orifice when the outlet (downstream) pressure is 52.8% or less of the inlet (upstream) pressure. Under the “choked flow” or “critical flow” condition, when the upstream gas pressure is maintained at a constant value, the mass flow rate of gas remains constant regardless of changes in outlet pressure. This means that a fixed mass flow rate of the calibration gases (i.e., gas 146.1 and gas 146.2) can be delivered regardless of lot-to-lot or unit-to-unit variation in shunt sensors 145. This allows consistent calibration without the use of active electronic flow controllers.

In contrast, some conventional systems use an adjustable orifice to set the outlet flow rate of the calibrator. An alternate approach could use mass flow controllers to maintain a constant flow rate regardless of changes in outlet pressure. A mass flow controller uses an adjustable orifice (i.e., valve) to maintain a consistent the flow rate across changes in outlet pressure. This approach typically would demand higher component costs and greater system complexity.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

What is claimed is:

1. A calibration system for a blood parameter monitoring system, the calibration system comprising:
a calibration device;
a first canister configured to fluidly couple with the calibration device and containing a first calibration gas having a first gaseous mixture;
a second canister configured to fluidly couple with the calibration device and containing a second calibration gas having a second gaseous mixture that is different than the first gaseous mixture; and
a computer-readable storage medium comprising instructions that, when executed, cause the calibration system to perform a two-point gas calibration process that comprises:
exposing, by the calibration device, a blood parameter sensor to a flow of the first calibration gas, and
ceasing, by the calibration device, the flow of the first calibration gas when the first output signal from the blood parameter sensor reaches a state of equilibrium.

2. The calibration system of claim 1, wherein the state of equilibrium of the first output signal from the blood parameter sensor is reached when the first output signal varies by less than a threshold amount over a set time period.

3. The calibration system of claim 1, wherein the flow of the first calibration gas is directed to flow through a fixed-size orifice of the calibration device.

4. The calibration system of claim 1, wherein the two-point gas calibration process further comprises:
exposing the blood parameter sensor to a flow of the second calibration gas comprising oxygen and carbon dioxide; and
ceasing the flow of the second calibration gas when the second output signal from the blood parameter sensor reaches the state of equilibrium.

5. The calibration system of claim 4, wherein the state of equilibrium of the first output signal from the blood parameter sensor is reached when the first output signal varies by less than a threshold amount over a set time period, and wherein the state of equilibrium of the second output signal from the blood parameter sensor is reached when the second output signal varies by less than the threshold amount over the set time period.

6. The calibration system of claim 4, wherein the two-point gas calibration process further comprises:
venting, by the calibrator device, the first calibration gas after ceasing the flow of the first calibration gas and prior to exposing the blood parameter sensor to the flow of the second calibration gas.

7. The calibration system of claim 4, wherein the first and second calibration gases are directed to flow through a fixed-size orifice of the calibration device.

8. A calibration process for a blood parameter monitoring system to improve blood parameter measurement accuracy, the calibration process comprising:
exposing a blood parameter sensor to a flow of a first calibration gas comprising oxygen and carbon dioxide,
wherein the flow of the first calibration gas: (i) passes through an orifice having a fixed size and (ii) has a pressure at an outlet of the orifice that is 52.8% or less than a pressure at an inlet of the orifice.

9. The calibration process of claim 8, further comprising adjusting the pressure of the first calibration gas at the inlet of the orifice to achieve the pressure at the outlet of the orifice that is 52.8% or less than the pressure at the inlet of the orifice.

10. The calibration process of claim 8, further comprising:
monitoring a first output signal from the blood parameter sensor while exposing the blood parameter sensors to the flow of the first calibration gas; and
ceasing the flow of the first calibration gas when the first output signal from the blood parameter sensor reaches a state of equilibrium.

11. The calibration process of claim 10, wherein the state of equilibrium of the first output signal from the blood parameter sensor is reached when the first output signal varies by less than a threshold amount over a set time period.

12. The calibration process of claim 8, further comprising:
exposing the blood parameter sensor to a flow of a second calibration gas comprising oxygen and carbon dioxide,
wherein the flow of the second calibration gas: (i) passes through an orifice having a fixed size and (ii) has a pressure at an outlet of the orifice that is 52.8% or less than a pressure at an inlet of the orifice.

13. The calibration process of claim 12, further comprising:
monitoring a first output signal from the blood parameter sensor while exposing the blood parameter sensors to the flow of the first calibration gas; and
ceasing the flow of the first calibration gas when the first output signal from the blood parameter sensor reaches a state of equilibrium;
monitoring a second output signal from the blood parameter sensor while exposing the blood parameter sensors to the flow of the second calibration gas; and
ceasing the flow of the second calibration gas when the second output signal from the blood parameter sensor reaches the state of equilibrium.

14. The calibration process of claim 8, wherein the state of equilibrium is reached when the first or second output signal varies by less than a threshold amount over a set time period.

15. A calibration system for a blood parameter monitoring system, the calibration system comprising:
a calibration device;
a first canister configured to fluidly couple with the calibration device and containing a first calibration gas having a first gaseous mixture;
a second canister configured to fluidly couple with the calibration device and containing a second calibration gas having a second gaseous mixture that is different than the first gaseous mixture; and
a computer-readable storage medium comprising instructions that, when executed, cause the calibration system to perform a two-point gas calibration process that comprises:
automatically initiating and running one or more self-diagnostic tests of the calibration system; and
in response to passing the one or more self-diagnostic tests, exposing a blood parameter sensor to a flow of the first calibration gas.

16. The calibration system of claim 15, wherein the one or more self-diagnostic tests comprise one or more of: a leak rate test, a valve operation test, and measuring a flow rate of the first calibration gas.

17. The calibration system of claim 15, wherein the two-point gas calibration process further comprises:
after exposing the blood parameter sensor to the flow of the first calibration gas, exposing the blood parameter sensor to a flow of a second calibration gas.

18. The calibration system of claim 17, wherein the two-point gas calibration process further comprises:
monitoring a first output signal from the blood parameter sensor while exposing the blood parameter sensors to the flow of the first calibration gas; and
ceasing the flow of the first calibration gas when the first output signal from the blood parameter sensor reaches a state of equilibrium;
monitoring a second output signal from the blood parameter sensor while exposing the blood parameter sensors to the flow of the second calibration gas; and
ceasing the flow of the second calibration gas when the second output signal from the blood parameter sensor reaches the state of equilibrium.

19. The calibration system of claim 17, wherein a composition of the first calibration gas is different than a composition of the second calibration gas.

20. The calibration system of claim 17, wherein the first and second calibration gases are directed to flow through a fixed-size orifice of the calibration device.
